(19) United States
(12) Patent Application Publication
(10) Pub. No.: US 2025/0206658 A1
(43) Pub. Date: Jun. 26, 2025

(54) METHOD FOR PRODUCING A DENTAL RESTORATION HAVING A TARGET COLOR VALUE

(71) Applicant: VITA ZAHNFABRIK H. RAUTER GMBH & CO. KG, Bad Säckingen (DE)

(72) Inventors: Michael GOEDIKER, Bad Säckingen (DE); Berit GOEDIKER, Bad Säckingen (DE)

(21) Appl. No.: 18/851,447

(22) PCT Filed: Apr. 26, 2023
(86) PCT No.: PCT/EP2023/060909

§ 371 Date: Sep. 26, 2024

(30) Foreign Application Priority Data
Apr. 27, 2022 (EP) 22170330.9

(51) Int. Cl. C03C 8/16; A61C 13/08; C03C 4/00; C03C 4/02

(52) U.S. Cl. CPC C03C 8/16; A61C 13/082; C03C 4/0021; C03C 4/02; C03C 2204/00; C03C 2209/00

(57) ABSTRACT

The present invention relates to a method for producing a dental restoration, the final coloring being obtained by glazing a molding with a colored glazing of a defined color value and heat-treating the glazed molding.

The present invention relates to a process for producing a dental restoration, in which the coloring is achieved by glazing a molded part with a colored glaze having a defined color value, and thermal treatment of the glazed molded part.

The term “restorative dental medicine” designates the treatments of dental problems with simultaneously restoring the teeth under functional and aesthetic aspects. In particular, the focus is on the natural adaptation of the teeth, which in many cases decides on the aesthetic acceptance of a dental restoration. For this purpose, a number of basic colors are usually available, which are adapted in color to the existing dental situation.

The conventional determination of the color of teeth is usually effected by means of so-called color rings or scales, which are provided with color specimens and serve for visual color comparison, in which the collection of color specimens traditionally corresponds to the tooth colors that occur most frequently in nature, which were determined empirically.

In this context, DE 693 19 291 describes a color mixing indicator device, which is employed in the selection of desirable artificial tooth colors in the production of bridges, crowns, partial dentures, and the like. A set of dental color mixture indicator devices is disclosed, each said component indicator device having a plurality of color samples located in an array and arranged about a central one of the color samples, with each sample spaced apart from samples adjacent thereto, the colors of said samples being representative of colors of a predetermined color system having a three-dimensional co-ordinate system (L, a, b) aligned with said array, wherein the color of each of said samples corresponds to the color of the corresponding location in said co-ordinate system (LO, a, b), the colors of the samples of each said component device being located on a plane of said co-ordinate system and the plane corresponding to each device being substantially parallel to, and spaced from, the planes of the other ones of said component devices, and whereby, in use of the said set, a patient's tooth can be compared against any one of said component devices and a desired dental color intermediate a selected pair of said color samples can be mixed for fabrication by a mixture of the coloring materials used to fabricate said selected pair, wherein the central color samples of adjacent planes are arranged along a common line, and offset with respect to an axis (L) of the co-ordinate system, which are perpendicular to mutually adjacent planes.

Such color scales are used for the stepwise determination of the tooth basic color according to the three color dimensions brightness, color intensity, and hue, which determine the human color impression. Corresponding color scales are commercially available, for example, under the designation of VITA SYSTEM 3D-MASTER® (cf. FIG. 2).

In order to achieve a more objective detection of tooth color, a number of technically supported methods are proposed in the prior art.

EP 3 643 272 A1 describes a process for determining a tooth color of a filling or another restoration is provided which in particular is produced using composite materials, wherein at least one tooth is scanned or is visually detected. The process is characterized in that natural teeth are spectrally measured in advance, that the color values measured within a color space are classified into several, in particular four, categories which each form a color cloud extending in a three-dimensional fashion within the color space, which is done with the help of a structuring algorithm, in particular a Nearest Neighbour Algorithm, or a symmetry recognition algorithm, and that the tooth color of the filling is determined by means of evaluating to which category the tooth scanned has the smallest color distance, or to which color cloud the tooth scanned belongs.

WO 2020/156855 discloses an assistance system for dental treatment, in particular for changing a tooth color, the system comprising: a measuring device configured to measure tooth data associated with teeth to be treated, the tooth data including at least one of a tooth color, a tooth coordinate, or any combination thereof; a data processing means, and a camera configured to capture a tooth image, wherein the data processing means comprises a receiving module, and the measuring device comprises a transmitting module configured to transmit measured tooth data from the measuring device to the data processing means; an allocation module integrated in the data processing means, the allocation module configured to allocate received tooth data to a tooth image; a selection means configured to select a level of color change; and an adaptation means configured to adapt the tooth image to the selected level of color change.

EP 3 650 821 relates to a system for determining the color of teeth, which comprises a 2D or 3D camera, wherein said system is adapted for acquiring images recorded by the camera, and an attachment, in which said attachment comprises a spacer that has a connecting means for attaching the attachment to the camera, in which said spacer includes an aperture by means of which the camera is adapted to record an image of teeth, in which said spacer is adapted to generate a distance between a recording device of the camera and the tooth surface, and in which said spacer is adapted to generate a distance between said recording device of the camera and the aperture, in which the camera has a normal recording mode without using the attachment, and a color measuring mode for use with an attachment for measuring the color of the tooth, in which the system for recognizing the presence of the attachment on the camera is adapted on the basis of the changes in the field of view recorded by the camera to switch to a color measuring mode when such changes are detected in the field of view.

WO 2020/154450 describes a method for color customization, comprising the following steps: providing tooth measurement data, wherein the tooth measurement data comprise at least one insertion area in which the dental restoration is to be inserted; providing restoration data; providing information about staining or glazing colors; generating a color characteristic from the tooth measurement data taking into account the restoration data, and calculating a multicolor target coloration from the color characteristic taking into account the information on staining or glazing colors, as well as output of the multicolor target coloration for color customization of the dental restoration.

DE 10 2015 204 109 describes a sprayable veneer ceramic composition for dental restorations of zirconium dioxide, comprising a disperse phase and a dispersion medium, in which said disperse phase contains a pulverized glass ceramic comprising SiO₂, Nb₂O₅, Al₂O₃, Li₂O, Na₂O and ZrO₂, and said dispersion medium contains isopropanol and/or ethanol and/or ethylene glycol, and in which said sprayable veneer ceramic composition contains at least one blowing agent. In order to optimize the aesthetics of the veneer ceramic, the pulverized glass ceramic may contain pigments as coloring or fluorescent additives.

US 2021/0322283 discloses a glazing composition for dental restorations made of zirconia. The glazing composition comprises a liquid, glass particles, and hydrophilic silica nano-particles.

U.S. Pat. No. 4,828,117 describes a multilayered dental restoration having a translucent porcelain layer overlying a more opaque ceramic layer, wherein the colors of the two layers match.

CN 108784875 relates to a color card sample made of zirconia.

WO 2020/185086 relates to a method for providing a natural color and optical depth to a dental object, in which the color and optical properties are oriented by those of a neighboring or adjacent tooth.

U.S. Pat. No. 5,906,490 provides a kit and method for the preparation of dental products, in which each of three materials having different lightness values is formed into layers of increasing opacity.

Both the conventional, i.e., the subjective-visual, determination of tooth colors and the technically supported objectified measurement of tooth colors are affected by many influence factors, so that the aesthetic result often depends on the skill and experience of the dentist and dental engineer. Further, the existing system has the drawback that a large number of samples must be stocked to achieve as color-true an adaptation as possible. In addition, in the cases where a wrong choice has been made, complicated color modifications and corrections have to be effected.

Thus, there is still a need for a simplified and reliable system for selecting and producing dental restorations having a natural appearance, which fit into the existing dental scheme. In addition, it would be desirable to be able to perform a simple adaptation of the tooth color.

Before this background, the present invention provides a method for preparing a dental restoration that allows for a simple and efficient color adaptation, and which can significantly reduce the number of samples that has to be stocked for an exact color matching.

Therefore, the present invention firstly relates to a method for producing, especially for color-matching, a dental restoration, comprising the following steps:
a) providing a molded part for dental restorations, in which said molded part has a basic color value FW(F), and wherein the basic color value FW(F) of the molded part has a maximum difference value ΔE of 5 from a target color value FW(Z) of the dental restoration;
b) applying a colored glaze to the molded part, wherein said glaze has a defined color value FW(Gₓ); and
c) heat-treating said glazed molded part to obtain the dental restoration,
characterized in that said color value FW(Gₓ) of the glaze corresponds to the difference ΔE between the color values FW(Z) and FW(F).

The color values and difference values are CIEL*a*b* values, wherein preferably the difference relates to one of the values at a time. The determination of such values is known to those skilled in the art, and has been described, for example, by A. Baltzer and V. Kaufmann-Jinoian in “Die Bestimmung der Zahnfarben”, issued in QZ Quintessenz Zahntechnik, volume 30, July 2004.

While conventional methods for producing dental restorations rely on the experience of the dental engineer and the color matching of the restoration is mostly done manually, the method according to the invention relies on a selected choice of glazes with a defined color value, which corresponds to the difference between the basic color value and the target color value of the molded part, so that the empirical procedure underlying the conventional method can be omitted. Rather, a selective matching of the molded part is enabled by purposefully selecting a glaze of suitable color. Therefore, in a preferred embodiment, the glaze having a defined color value FW(Gₓ) is provided by selecting from a number of glazes having defined color values FW(G).

The glaze is preferably selected to have a color value FW(Gₓ) that corresponds to the difference between the basic color value FW(F) of the molded part and the target color value FW(Z) of the molded part. Therefore, for example:

$$FW ( {Z}_{1} ) = FW ( {F}_{1} ) + FW ( {G}_{1} )$$

Expressed otherwise:

$$FW ( {G}_{x} ) = FW ( {Z}_{y} ) - FW ( {F}_{z} )$$

$$with$$

$$y = x + z$$

$$and$$

$$y - z < 5$$

In the cases where a matching tooth color is not available or a wrong choice has been made by the user, manual color corrections are often necessary. Thus, at first, the appropriate ceramic painting color has to be applied with a paintbrush or spray to the right place, which requires a certain experience. This is followed by a so-called fixing firing of stains. Subsequently, a glaze is applied, and glaze firing is effected, and only then can the color effect be definitely evaluated. Because of the method according to the invention, the color matching can be done with only one firing, whereby the result can be evaluated more quickly.

Unlike conventional veneers, which are usually colorless and are employed for achieving a particular gloss, the method according to the invention uses colored glazes, in which the coloring is preferably effected by colored pigments. Therefore, in a preferred embodiment of the process according to the invention, glazes are used that have a liquid mixing phase, and a solid phase including coloring substances, preferably colored pigments, wherein the proportion of the liquid mixing phase in the glaze is at least 60% by weight, based on the total weight of the glaze.

In this way, a selection of glazes having a defined color value can be provided, from which the glaze is selected whose color value corresponds to the difference between the basic color value of the molded part and the target color value to be achieved. Thus, on the one hand, the step of empirically mixing the glaze can be omitted, and on the other hand, the target color value is achieved by applying only one glaze.

Within the scope of the method according to the invention, the provision of the molded part is preferably effected by selecting from a set of molded parts each having a defined basic color value FW(F) by comparing with the teeth neighboring the restoration in the patient's mouth, preferably by using a color scale for determining the tooth colors. This type of providing in combination with the color adaptation by applying a selected glaze enables the stocking of a limited selection of different basic colors, whose color can be adapted by a corresponding glaze. Thus, the necessity of stocking a complete color assortment of blanks no longer exists. Rather, it is now possible to achieve particular color locations by means of different colored glazes starting from defined starting points. Therefore, said basic color value and said target color value are preferably defined color values as defined in a standardized color scale for determining tooth colors. Such standardized color scales are known to those skilled in the art and are commercially available, for example, under the designation of VITA classical A1-D4®, VITA SYSTEM 3D-MASTER®, or VITA Toothguide 3D-MASTER® (cf. also FIG. 2).

In order to minimize the influence of the subjective perception of colors in the color selection, it has proven advantageous to rely on digital support. Therefore, an embodiment of the process according to the invention is preferred in which the determination of the ΔE and/or the selection of the molded part is effected with computer assistance.

In accordance with the process according to the invention, the target color value FW(Z) of the dental restoration is achieved by applying a corresponding glaze. It has proven advantageous to limit the thickness in which the glaze is applied to thus minimize adverse affection of the color result. Therefore, in a preferred embodiment of the process according to the invention, the glaze is applied to the molded part in a thickness of from 10 to 50 μm, preferably from 20 to 40 μm. The application of the glaze can be effected by methods known to those skilled in the art manually or by machine.

The method according to the invention used glazes with defined color values in order to achieve an adaptation of the color of a provided molded part. Therefore, the present invention further relates to a glaze for use in the method according to the invention, in which said glaze has
i) a liquid mixing phase; and
ii) a solid phase including coloring substances, preferably colored pigments,
wherein the proportion of the liquid mixing phase in the glaze is at least 60% by weight, based on the total weight of the glaze, and wherein said glaze has a defined color value FW(Gₓ).

The defined color value FW(Gₓ) of the glaze corresponds to the difference ΔE between the basic color value FW(F) of a molded part and the target color value FW(Z) of a dental restauration to be produced from such molded part.

The mixing phase is preferably selected from the group consisting of water, one or more alcohols, and mixtures thereof. Said alcohol is preferably selected from the group of monohydric, dihydric and trihydric alcohols, and mixtures thereof. In a particularly preferred embodiment, said alcohol is selected from the group consisting of glycerol, 1,3-butanediol, 1,2-propanediol, ethanol, and mixtures thereof.

In a preferred embodiment, the mixing phase further includes one or more complexing agents, preferably selected from the group consisting of ethylenediaminetetraacetic acid, and citric acid.

As a mixing phase, those that are commercially available, such as Triplex® solution B, can be used.

As the solid phase of the glaze, compositions known in the prior art can be used. Preferably, a basic mixture is employed as the solid phase of the glaze, to which coloring substances, especially colored pigments, are admixed depending on need. In a preferred embodiment, the solid phase of the glaze includes from 45 to 70% by weight SiO₂, more preferably from 50 to 65% by weight, based on the total weight of the solid phase. Further preferred is an embodiment in which the solid phase includes one or more of the following components, respectively based on the total weight of the solid phase:
5 to 15% by weight, preferably 7 to 13% by weight, of Al₂O₃;
5 to 15% by weight, preferably 7 to 13% by weight, of B₂O₃;
5 to 15% by weight, preferably 7 to 13% by weight, of K₂O;
5 to 15% by weight, preferably 7 to 13% by weight, of Na₂O.

In order to achieve the desired color values, the solid phase may include up to 5% by weight of coloring substances, especially colored pigments, based on the total weight of the solid phase.

Pigments and pigment mixtures that are common in the branch, as used in the ceramics, glass and porcelain industries, may be employed as said coloring substances. In this respect, the glaze according to the invention has a high compatibility with existing systems.

In a further preferred embodiment, the glaze is preferably in the form of a kit, comprising a container with the mixing phase, and a container with the solid phase comprising coloring substances. This form of provision has the advantage that the desired glaze can be mixed on site, and drying out from storage can be prevented.

It is an object of the present invention to simplify the production of dental restorations with desired color values. In this context, the present invention further relates to a kit for the production of a dental restoration, comprising a) an assortment of glazes for molded parts for dental restorations, wherein each glaze has a defined color value FW(Gₓ), in which the color values FW(Gₓ) correspond to the differences ΔE between the color samples of a standardized color scale for tooth color determination, in which ΔE is not higher than 5, and b) a color guide for assigning an established ΔE value to the corresponding glaze.

The kit according to the invention is preferably employed in the method according to the invention, allowing to achieve a dental restoration with a target color value FW(Z) in a simple way and with only one heat treatment, starting from a molded part with a basic color value FW(F) by selecting a suitable glaze, in which the color value FW(G) of the glaze corresponds to the difference ΔE between the basic color value FW(F) of the molded part and the target color value FW(Z) of the dental restoration. The kit according to the invention enables a specific selection of the suitable glaze, which corresponds to the established color value difference ΔE between the basic color value FW(F) and the target color value FW(Z), by providing a color guide. Thus, the color difference can be balanced by applying the glaze and thermal treatment, and a dental restoration can be made available that is aesthetically included in the patient's existing tooth scheme.

A number of standardized color scales employed for determining tooth colors are available to those skilled in the art. Preferably, said standardized color scale, which may be part of the kit according to the invention, is VITA classical A1-D4®, VITA SYSTEM 3D-MASTER®, or VITA Toothguide 3D-MASTER®.

In a preferred embodiment, the kit according to the invention further includes a standardized color scale for determining tooth colors.

The kit according to the invention enables a broad color spectrum to be achieved by using a limited number of glazes. In a preferred embodiment, the kit includes an assortment of 2 or more, preferably 3 or more, more preferably 4 or more, glazes and/or not more than 10, preferably not more than 8, more preferably not more than 6, glazes.

In a further preferred embodiment, the kit further includes an applicator for applying the glaze to a molded part for dental restorations. In an also preferred embodiment, in addition to the assortment of glazes, the color guide and optionally the applicator, the kit according to the invention further includes an assortment of molded parts for dental restorations each having ma basic color value FW(Fₓ), which preferably correspond to the color values of a standardized color scale for determining tooth colors. In this way, a complete package can be made available that allows for a simple and efficient preparation of dental restorations.

Preferably, the glazes of the kit according to the invention are those according to the present invention.

The present invention further relates to the use of a glaze with a defined color value FW(Gₓ) in the production of dental restorations, in which the color value FW(Gₓ) corresponds to the difference ΔE between the color samples of a standardized color scale for tooth color determination, in which ΔE is not higher than 5.

FIG. 1 and the following Example illustrate the present invention, but are by no means to be understood as limiting the concept of the invention.

FIG. 1 illustrates the concept according to the invention in which, starting from a dental restoration, for example, as commercially available and having a particular color value FW(Fₓ), such color value can be adapted to a desired target color value FW(Zₓ). In particular, the target color value is obtained by comparing with a patient's existing tooth situation, and takes the patient's individual tooth coloring into account. Therefore, a colored glaze whose color value FW(Gₓ) corresponds to the difference between the color value FW(Fₓ) of the starting restoration and the desired target color value FW(Zₓ) is applied to the starting restoration.

FIG. 2 shows a typical color scale for determining and selecting dental molded parts (VITA Toothguide 3D-MASTER®).

Starting from a ceramic restoration with a basic color value, for example, FW(F₁), the present invention enables a simple and efficient color adaptation to a target color value FW(Z₁) by applying a glaze whose color value FW(Gₓ) corresponds to the difference between the color values FW(F₁) and FW(Z₁). Depending on requirements, this may be done in one step or several steps. The selection of suitable glazes is effected by means of an acknowledged color value scale, taking the hue and the color intensity into account.

A crown having a color value FW(F₂), which corresponds to the designation 3M2 from the color scale VITA SYSTEM 3D-MASTER®, serves as the starting molded part. This starting molded part is to be adapted in color to correspond to the target value FW(Z₅) (see FIG. 1), which corresponds to the tooth color 3M3 from the color scale VITA SYSTEM 3D-MASTER® in this Example. In order to achieve this, a colored glaze is selected and applied, whose color value corresponds to the difference between the starting color value and the target color value for an application thickness of about 30 μm. Table 1 illustrates the procedure according to the invention:

TABLE 1

| | Color | Transmission | | | | | | | |
|---|---|---|---|---|---|---|---|---|---|
| Product | value | [%] | L* | a* | b* | ΔE* | ΔL | Δa | Δb |
| Starting | FW(2) | | 76.30 | 3.36 | 20.24 | | | | |
| crown | | | | | | | | | |
| colored | FW(G5) | 43.31 | 74.43 | 3.33 | 23.76 | | | | |
| glaze * | | | | | | | | | |
| Target | FW(Z5, | | 76.40 | 3.80 | 22.50 | 2.27 | −0.10 | −0.17 | −2.26 |
| color | target) | | | | | | | | |
| location | | | | | | | | | |
| Crown + | FW(Z5, | | 76.48 | 3.70 | 21.88 | 0.63 | 0.08 | −0.10 | −0.62 |
| Glaze | actual | | | | | | | | |
| | value) | | | | | | | | |

* The color was measured in transmission because the glaze is a translucent glaze.

Because of the higher opacity, the color values of the crowns were measured in reflection.

As can be seen from the values in Table 1, a good matching with the desired target color value is achieved by applying the colored glaze to the starting crown.

1. A method for producing a dental restoration, comprising the following steps:
a) providing a molded part for dental restorations, in which said molded part has a basic color value FW(F), and wherein the basic color value FW(F) of the molded part has a maximum difference value ΔE of 5 from a target color value FW(Z) of the dental restoration;
b) applying a glaze to the molded part, wherein said glaze has a defined color value FW(Gₓ);
c) heat-treating said glazed molded part to obtain the dental restoration, characterized in that the defined color value FW(Gₓ) of the glaze corresponds to the difference ΔE between the color values FW(Z) and FW(F), said color values being CIEL*a*b* values.

2. The method according to claim 1, characterized in that the glaze having a defined color value is provided by selecting from a number of glazes having defined color values FW(G).

3. The method according to claim 1, characterized in that the molded part is provided by selecting from a set of molded parts each having a defined basic color value FW(F) by comparing with the teeth neighboring the restoration in the patient's mouth by using a color scale.

4. The method according to claim 1, characterized in that the basic color values are those that correspond to the color values in the standardized color scales for tooth color determination.

5. The method according to claim 1, characterized in that the determination of the ΔE and/or the selection of the molded part is effected with computer assistance.

6. The method according to claim 1, characterized in that the glaze is applied to the molded part in a thickness of from 10 to 50 μm.

7. A glaze for use in a method according to claim 1, characterized in that the glaze has
i) a liquid mixing phase; and
ii) a solid phase including coloring substances
wherein the proportion of the liquid mixing phase in the glaze is at least 60% by weight, based on the total weight of the glaze, and wherein said glaze has a defined color value FW(Gₓ).

8. The glaze according to claim 7, characterized in that the liquid mixing phase is selected from the group consisting of water, one or more alcohols, and mixtures thereof, in which said alcohol is selected from the group of monohydric, dihydric and trihydric alcohols, and mixtures thereof.

9. The glaze according to claim 7, characterized in that the liquid mixing phase further includes one or more complexing agents.

10. The glaze according to claim 7, characterized in that said glaze is in the form of a kit, said kit comprising a first container including the liquid mixing phase, and a second container including the solid phase with coloring substances.

11. A kit for producing a dental restoration, including
a) an assortment of glazes for molded parts for dental restorations, wherein each glaze has a defined color value F(Gₓ), in which the color values F(Gₓ) correspond to the differences ΔE between the color samples of a standardized color scale for tooth color determination, and in which ΔE is not higher than 5; and
b) a color guide for assigning an established ΔE value to the corresponding glaze.

12. The kit according to claim 11, characterized in that said assortment includes 2 or more glazes and/or not more than 10, glazes.

13. The kit according to claim 11, characterized in that the kit further includes an applicator for applying the glaze to a molded part for dental restorations.

14. The kit according to claim 11, characterized in that said glazes have
i) a liquid mixing phase; and
ii) a solid phase including coloring substances
wherein the proportion of the liquid mixing phase in the glaze is at least 60% by weight, based on the total weight of the glaze, and wherein said glaze has a defined color value FW(Gₓ).

15. A method for producing dental restorations, the method comprising using a glaze with a defined color value FW(Gₓ) in the production of dental restorations, in which the color value FW(Gₓ) corresponds to the difference ΔE between the color samples of a standardized color scale for tooth color determination, and in which ΔE is not higher than 5.

16. The method according to claim 6, characterized in that the glaze is applied to the molded part in a thickness of from 20 to 40 μm.

17. The glaze according to claim 8, characterized in that the alcohol is selected from the group consisting of glycerol, 1,3-butanediol, 1,2-propanediol, ethanol, and mixtures thereof.

18. The glaze according to claim 9, wherein the complexing agents is selected from a group consisting of ethylenediaminetetraacetic acid and citric acid.

19. The kit according to claim 12, characterized in that said assortment includes 3 or more glazes and/or not more than 8 glazes.

20. The kit according to claim 19, characterized in that said assortment includes 4 or more glazes and/or not more than 6 glazes.
